(19) United States
(12) Patent Application Publication
(10) Pub. No.: US 2018/0037908 A1
(43) Pub. Date: Feb. 8, 2018

(54) SYSTEM AND METHOD OF COMBINING GENETIC MATERIAL FROM DISTINCT SPERM CELLS TO CREATE ONE NEW SPERM CELL WITH THE COMBINED GENETIC MATERIAL

(71) Applicant: Leigh M. Rothschild, Sunny Isles Beach, FL (US)

(72) Inventor: Leigh M. Rothschild, Sunny Isles Beach, FL (US)

(73) Assignee: Leigh M. Rothschild, Miami, FL (US)

(21) Appl. No.: 15/228,028

(22) Filed: Aug. 4, 2016

(51) Int. Cl. C12N 15/85; A61B 17/435

(52) U.S. Cl. CPC C12N 15/85; A61B 17/435

(57) ABSTRACT

A method of creating a sperm cell comprising combined genetic material of human males is disclosed. Deoxyribonucleic Acid (DNA) is extracted from a living cell, of a first male. A gene sequence is separated from the Deoxyribonucleic Acid (DNA) of the first male. A recombinant Deoxyribonucleic Acid (DNA) is generated by inserting and attaching the gene sequence of the first male into a Deoxyribonucleic Acid (DNA) of a living cell of a second male. The recombinant Deoxyribonucleic Acid (DNA) is programmed to create Induced pluripotent stem cells (iPSCs). The Induced pluripotent stem cells (iPSCs) are converted into at least one recombinant sperm cell comprising combined genetic material of the first male and the second male.

### CROSS-REFERENCE TO RELATED PATENT APPLICATION

The present application is related to and claims priority of U.S. provisional patent application titled “System and method of combining genetic material from distinct sperm cells to create one new sperm cell with the combined genetic material”, Ser. No. 62/241,766, filed on Oct. 15, 2015, the description of the same is incorporated herein in its entirety.

### FIELD OF THE DISCLOSURE

The presently disclosed embodiments are generally related to recombination of genetic materials and more particularly to creating a recombinant sperm cell comprising combined genetic materials of human males.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

During reproduction in humans, a male sperm fertilizes a female egg to develop an embryo or a zygote. The embryo further leads to the development of a human offspring. During fertilization, the sperm passes its entire genetic information to the egg. Thus, the zygote shares genetic information of a male and a female.

The world has seen numerous cases of same-sex relationships and marriages. A marriage between two persons of same sex has not been legally validated hitherto. However, in recent times, several jurisdictions are providing a legal status to such marriages. In scenarios where the marriage involves two male partners, child bearing is possible only if a child is adopted or by conceiving an offspring between a surrogate female and one of the male partners. A surrogate female carries an embryo in her womb to give birth to a child. The embryo develops upon in-vitro fertilization of a sperm cell with an egg. In such scenarios the problem that arises is, a sperm cell of only one of the male partner can used to fertilize the egg. Consequently, the child carries genetic structure of only one male, whose sperm cell is used to fertilize the egg. Thus, it is not possible for both males to provide their genetic traits to a common offspring.

### BRIEF SUMMARY

It will be understood that this disclosure in not limited to the particular devices, and methodologies described, as there can be multiple possible embodiments of the present disclosure which are not expressly illustrated in the present disclosure. It is also to be understood that the terminology used in the description is for the purpose of describing the particular versions or embodiments only, and is not intended to limit the scope of the present disclosure.

In an example embodiment, a method of creating a sperm cell comprising combined genetic material of males is described. The method comprises extracting Deoxyribonucleic Acid (DNA) from a living cell, of a first male. The method further comprises separating a gene sequence from the Deoxyribonucleic Acid (DNA) of the first male. The method further comprises generating a recombinant Deoxyribonucleic Acid (DNA) by inserting and attaching the gene sequence of the first male into a Deoxyribonucleic Acid (DNA), of a living cell, of a second male. The method further comprises creating Induced pluripotent stem cells (iPSCs) by programming the living cell of the second male, comprising the recombinant Deoxyribonucleic Acid (DNA). The method further comprises converting the Induced pluripotent stem cells (iPSCs) into at least one recombinant sperm cell. The at least one recombinant sperm cell comprises combined genetic material of the first male and the second male.

In another example embodiment, a method of creating a sperm cell comprising combined genetic material of males is described. The method comprises extracting Deoxyribonucleic Acid (DNA) from a living cell, of a first male. The method further comprises separating a gene sequence from the Deoxyribonucleic Acid (DNA) of the first male. The method further comprises extracting stem cells from a second male. The method further comprises introducing the gene sequence of the first male into a Deoxyribonucleic Acid (DNA), of the stem cells of the second male. The method further comprises converting the stem cells into at least one recombinant sperm cell. The at least one recombinant sperm cell comprises combined genetic material of the first male and the second male.

In yet another example embodiment, a method of creating a sperm cell comprising combined genetic material of males is described. The method comprises extracting Deoxyribonucleic Acid (DNA) from a living cell, of a first male. The method further comprises separating a gene sequence from the Deoxyribonucleic Acid (DNA) of the first male. The method further comprises introducing the gene sequence of the first male into a Deoxyribonucleic Acid (DNA), of sperm cells of the second male to create at least one recombinant sperm cell. The at least one recombinant sperm cell comprises combined genetic material of the first male and the second male.

### BRIEF DESCRIPTION OF THE DRAWINGS

The accompanying drawings illustrate various embodiments of systems, devices, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a flow chart that shows a method of creating a sperm cell comprising combined genetic material of human males, according to a first example embodiment.

FIG. 2 illustrates a flow chart that shows a method of creating a sperm cell comprising combined genetic material of human males, according to a second example embodiment.

FIG. 3 illustrates a flow chart that shows a method of creating a sperm cell comprising combined genetic material of human males, according to a third example embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The present disclosure may be implemented in form of multiple steps. Implementation of each step may require numerous biomedical devices and techniques. The biomedical devices may comprise a sterilization device, gene gun, microscope, DNA analyzer, assisted reproduction needle, assisted reproduction catheters, cannulae, introducer, dilator, sheath, stylet, powered aspiration pump, syringe pumps, embryo incubator, cryopreservation device, pH meter, and other medical devices used for different medical purposes falling within a scope of the present disclosure.

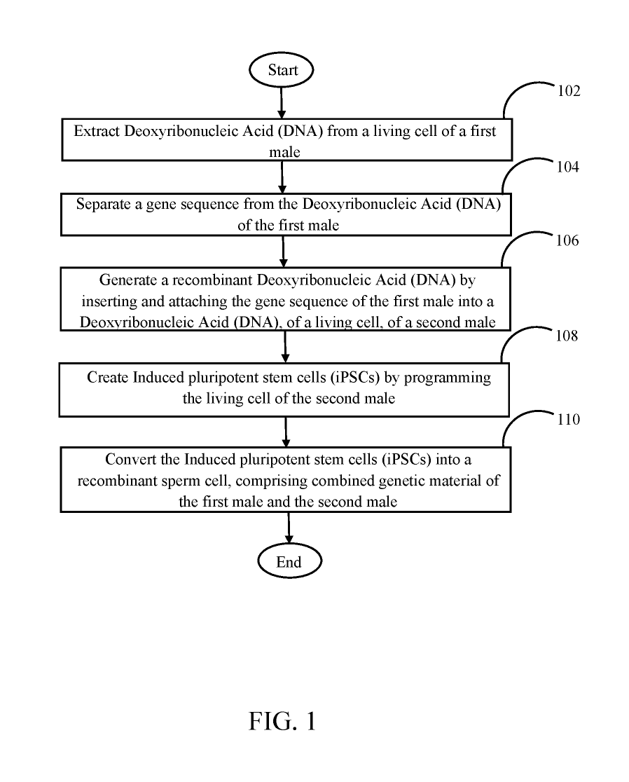

It is an object of the current disclosure to provide a method of creating a sperm cell comprising combined genetic material of males. The method of creating a sperm cell comprising combined genetic material may now be described referring to an exemplary embodiment shown in FIG. 1. A head of a sperm cell may comprise a nucleus. The nucleus may comprise a set of chromosomes. Each chromosome of the set of chromosomes may comprise Deoxyribonucleic Acid (DNA). The Deoxyribonucleic Acid (DNA) may comprise genetic information of an organism. In the Deoxyribonucleic Acid (DNA), the genetic information may be present in form of amino acid sequences. The amino acid sequences may be made up of adenine, guanine, cytosine, and thymine as constituent amino acids.

For modifying any genetic information, a particular sequence of amino acids present in the Deoxyribonucleic Acid (DNA) may be modified. To modify the genetic information, a gene sequence of cells of a first male, in a male-male couple comprising a first male and a second male, may need to be extracted. In order to do so, cells from body of the first male may be collected. The cells may include, but are not limited to, cheek cells, blood cells, hair follicles, sperm cells, epidermis of skin, and any other living tissues of the body.

In a preferred embodiment, sperm cells of a first male may be collected to extract the Deoxyribonucleic Acid (DNA), as illustrated at step 102. Such collected sperm cells may be treated with a lysis buffer, for breaking down lipid molecules present in membranes of a nucleus of the sperm cells. Subsequently, the sperm cells may be treated with alcohol and a centrifuge is used upon a test tube containing the mixture. Upon centrifugation, Deoxyribonucleic Acid (DNA) settles down at a bottom of test tube and is thus separated from cell debris.

In another embodiment, other techniques may also be used for extracting the Deoxyribonucleic Acid (DNA) from living cells. Other techniques for Deoxyribonucleic Acid (DNA) extraction may include, but are not limited to, organic (phenol-chloroform) extraction, non-organic (proteinase K and salting out) extraction, chelex (ion-exchange resin) extraction, FTAÔ paper (collection, storage, and isolation), and silica based (silica exchange resin- qiagen) extraction method.

From the Deoxyribonucleic Acid (DNA), a particular gene sequence may be identified. In one exemplary embodiment, the gene sequence may be identified using Southern blotting technique. The gene sequences may correspond to various traits of an individual like an eye colour, hair colour, shape of nose, shape of ear lobes, height of an individual, skin pigmentation, and other physical traits. Upon identification, the gene sequences of the first male may be separated, as illustrated at step 104. In an exemplary embodiment, the gene sequences of the first male could be separated using restriction enzymes.

A Deoxyribonucleic Acid (DNA) strand of a second male may be cut to receive the gene sequence of the first male. The gene sequence of the first male may be inserted into the Deoxyribonucleic Acid (DNA) strand of the second male in the couple, as illustrated at step 106. The gene sequence may be introduced into Deoxyribonucleic Acid (DNA) structure of any living cell of the second male, kept in a culture.

For inserting the gene sequence of the first male, restriction sites may be created into the Deoxyribonucleic Acid (DNA) structure of the living cell of the second male. Subsequently, the gene sequence of the first male may be introduced at the restriction sites. In one exemplary embodiment, the gene sequence could be inserted and attached using a Deoxyribonucleic Acid (DNA) ligase enzyme. Thus, a recombinant Deoxyribonucleic Acid (DNA) or chimeric Deoxyribonucleic Acid (DNA) is obtained in the living cell of the second male.

Successively, the cell comprising the recombinant Deoxyribonucleic Acid (DNA) may be programmed to create Induced pluripotent stem cells (iPSCs), as illustrated at step 108. The Induced pluripotent stem cells (iPSCs) may be created using genes encoding transcription factors. The Induced pluripotent stem cells (iPSCs) possess the ability to differentiate into other living cells and other organs of a body i.e. heart, nervous system, respiratory system, etc. Thus, the Induced pluripotent stem cells (iPSCs) may be used to create a recombinant sperm i.e. a sperm cell comprising combined genetic material of the two males, as illustrated at step 110.

In another embodiment, Deoxyribonucleic Acid (DNA) may be extracted from a living cell of a first male, as illustrated at step 202. Subsequently, a gene sequence of the first male may be separated. The gene sequence may be separated from a Deoxyribonucleic Acid (DNA), as illustrated at step 204. Stem cells of a second male may then be extracted, as illustrated at step 206. The stem cells may be extracted from a spinal cord fluid or a bone marrow of the second male.

The gene sequence of the first male may then be introduced into the stem cells of the second male. The gene sequence may be introduced into a Deoxyribonucleic Acid (DNA), as illustrated at step 208. Thereafter, the stem cells may be converted into at least one recombinant sperm cell, as illustrated at step 210. The at least one recombinant sperm cell may comprise combined genetic material of the first male and the second male.

In yet another embodiment, the gene sequence separated from the Deoxyribonucleic Acid (DNA) of the first male may be introduced into sperm cells of the second male, as illustrated at step 302 and 304. The gene sequence may be inserted into a Deoxyribonucleic Acid (DNA) of the sperm cells to create at least one recombinant sperm cell, as illustrated at step 306. The at least one recombinant sperm cell may comprise combined genetic material of the first male and the second male.

Subsequently, a female egg maybe retrieved from a healthy female. The recombinant sperm may then be fertilized in-vitro with a female egg. In one case, a sperm solution comprising a plurality of the recombinant sperms may be added to the egg for natural occurrence of fertilization. In another case, Intra-Cytoplasmic Sperm Injection (ICSI) may be performed. During ICSI, genetic material of the sperm is injected into the female egg. Thus, in both of the cases, the sperm fertilizes with the egg.

Fertilization of the sperm with the female egg results in development of an embryo. The embryo may then be transferred to a uterus of a female to give birth to an offspring. The embryo sticks to placenta of the female and draws nourishment for self growth. This embryo upon further development becomes a child.

In one embodiment, multiple embryos may be transferred to the placenta of the female. These multiple embryos may develop into twins. Thus, such offspring developed in the above described manner carries genetic characteristics of both parents, the first male and the second male. The offspring may be genetically unrelated to the female (surrogate mother).

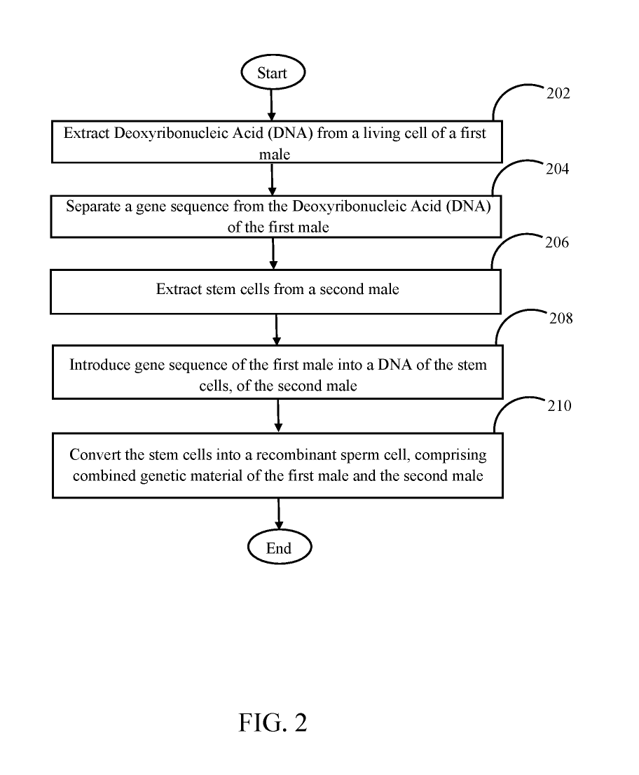

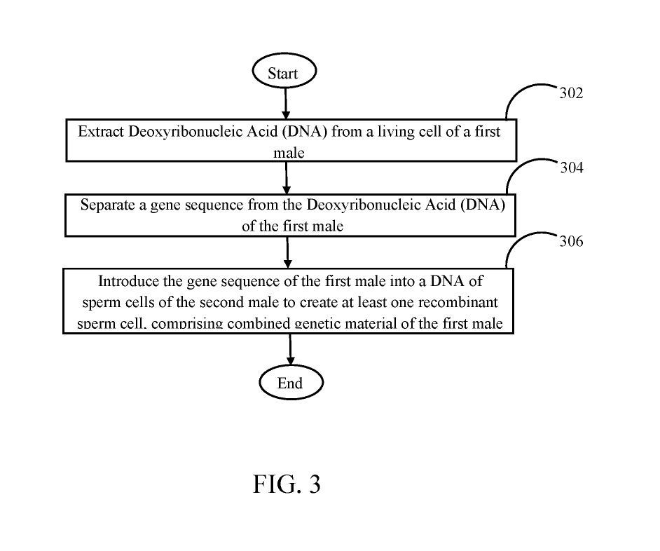

The flow chart of FIG. 1, FIG. 2, and FIG. 3 shows the method steps executed according to one or more embodiments of the present disclosure. It should also be noted that in some alternative implementations, the functions noted in the blocks may occur out of the order noted in the drawings. For example, two steps shown in succession in FIG. 1, FIG. 2, and FIG. 3 may in fact be executed substantially concurrently or the steps may sometimes be executed in the reverse order, depending upon the functionality involved. Any process descriptions or blocks in flow charts should be understood as steps in the process, and alternate implementations are included within the scope of the example embodiments in which functions may be executed out of order from that shown or discussed, including substantially concurrently or in reverse order, depending on the functionality involved.

Moreover, although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the device, process, composition of matter, means, methods and steps described in the specification. As one will readily appreciate from the disclosure, processes, devices, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, devices, manufacture, compositions of matter, means, methods, or steps.

What is claimed is:

1-13. (canceled)

14. A method of combining genetic material of a first human male and a second human male, the method comprising:
extracting Deoxyribonucleic Acid (DNA) from a living cell, of a first male;
separating a gene sequence from the Deoxyribonucleic Acid (DNA) of the first male;
introducing the gene sequence of the first male into a Deoxyribonucleic Acid (DNA), of sperm cells of the second male; and
extracting at least one recombinant sperm cell from the sperm cells of the second male having the gene sequence of the first male, wherein the at least one recombinant sperm cell comprises combined genetic material of the first male and the second male.

15. The method of claim 14, wherein the living cell of the first male is selected from a group consisting of cheek cells, blood cells, hair follicles, and epidermis of skin.

16. The method of claim 14, further comprising developing an embryo by in-vitro fertilization of the recombinant sperm with a female egg to develop an embryo.

17. The method of claim 14, further comprising performing Intra-Cytoplasmic Sperm Injection (ICSI) of the recombinant sperm with a female egg to develop an embryo.

18. The method of claim 16, further comprising placing the embryo in placenta of a female surrogate.

19. The method of claim 17, further comprising placing the embryo in placenta of a female surrogate.
